(19) United States
(12) Patent Application Publication
(10) Pub. No.: US 2015/0018593 A1
(43) Pub. Date: Jan. 15, 2015

(54) Catalyst, Process For The Preparation Of Said Catalyst And Use Of Said Catalyst In A Process And In A Device For The Preparation Of Olefins

(71) Applicant: Christian Trischler, Eggersdorf b. Graz (AT)

(72) Inventor: Heinrich Trischler, Linz (AT)

(21) Appl. No.: 14/382,764

(22) PCT Filed: Mar. 14, 2013
(86) PCT No.: PCT/EP2013/000781

§ 371 Date: Sep. 4, 2014

(30) Foreign Application Priority Data
Mar. 16, 2012 (AT) A327/2012
Apr. 24, 2012 (AT) A498/2012
Jun. 1, 2012 (AT) A635/2012

(51) Int. Cl. B01J 27/24; B01J 27/188; H01M 8/06; B01J 27/195; B01J 27/16; C07C 2/76; B01J 27/22; B01J 27/19

(52) U.S. Cl. CPC B01J 27/24; B01J 27/22; B01J 27/188; B01J 27/19; B01J 27/195; B01J 27/16; C07C 2/76; H01M 8/0612; C07C 2529/84
USPC 585/661; 502/177; 502/200; 422/187; 429/411

(57) ABSTRACT

The present invention relates to a catalyst characterized in that it comprises a) at least one metal compound selected from a group consisting of metal carbide, -nitride, -silicide, -phosphide and -sulfide or mixtures thereof, wherein the metal is selected from a group consisting of molybdenum, tungsten, tantalum, vanadium, titanium, niobium, lanthanum and chromium, and b) at least one non-Brønsted-acidic binder selected from a group consisting of AlPO₄, Bentonite, AlN and N₄Si₃. Furthermore, the present invention relates to a process or a device for the preparation of olefins from C2-, C3- or C4-alkanes using the catalyst.

The invention relates to a catalyst, a process for the preparation of said catalyst and the use of said catalyst in a process and in a device for the preparation of olefins.

Transition metal carbides, -phosphides, -nitrides, -silicides as well as -sulfides are refractory compounds, which are used for different applications.

For example, US-A-2010/02559831 describes the hydrogenation of cellulose to ethylene glycol in the presence of a tungsten carbide as catalytically active main component with additional parts of other transition metals like nickel, cobalt, iron, ruthenium, rhodium, palladium, osmium, platinum or copper. The catalyst is applied on a carrier material such as activated charcoal, aluminum oxide, silica, titanium oxide, silicon carbide or zirconium oxide. The catalyst is prepared by soaking the carrier material with salt solutions of the catalytically active components.

U.S. Pat. No. 5,321,161 describes the hydrogenation of nitriles to amines in the presence of a tungsten carbide catalyst, which is prepared by calcination of a tungsten salt with an acyclic compound, which contains a nitrogen-hydrogen bridge, such as guanidine.

U.S. Pat. No. 4,325,843 discloses a process for the preparation of a tungsten carbide catalyst, which is applied to a carrier. In this process, tungsten oxide is initially provided on a carrier and subsequently transformed into the nitride by heating in an ammonium atmosphere and finally transformed into the carbide by heating in a carbide atmosphere.

EP-A-1479664 describes the process for the preparation of olefins by metathesis in the presence of a carbide or oxycarbide of a transition metal, for example, tungsten and molybdenum. The preparation of the catalyst, which is applied to a carrier, is accomplished by soaking the carrier (for example, Al₂O₃, aluminum silicates, Ga₂O₃, SiO₂, GeO₂, TiO₂, ZrO₂ or SnO₂) with a solution of a transition element compound, subsequently, drying and calcinating the soaked carrier and finally tempering the soaked carrier at a temperature of 550 to 1000° C. in an atmosphere, which contains a hydrocarbon compound and hydrogen.

WO-A-2006/070021 describes a coating system, which comprises metal carbides and a binder, which at least partially contains an organic phosphate binder such as AlPO₄.

U.S. Pat. No. 3,649,523 describes a catalyst for hydrocracking of mineral oil fractions, which consists of a catalytically active metal oxide or -sulfide of cobalt, molybdenum, nickel or tungsten, a co-catalytic acidic carrier for hydrogen transfer, which contains aluminum silicates, as well as one additional porous material such as aluminum oxide, aluminum phosphate or silica. However, this publication does not disclose the cracking of low molecular weight alkanes such as C2-, C3- and C4-alkanes. Furthermore, it does not disclose a dehydrogenation reaction under formation of olefins.

Different processes for the dehydrogenation and cracking of C2-C4-alkanes are used. Commonly used catalysts for this reaction are, for example, Brønsted-acidic zeolites. The preparation of ethene and propene from propane and butane is conducted catalytically, for example, by using Brønsted-acidic zeolites ZSM5, ZSM23, and ZSM50 as described in U.S. Pat. No. 4,929,790, U.S. Pat. No. 4,929,791 and U.S. Pat. No. 5,159,127. In this process, coke and gasoline are formed as undesired side products, which lead to a loss of carbon and require frequent regeneration of the catalysts by burning off the formed coke under the formation of CO₂.

Lower olefins, such as ethene and propene, can also be prepared by steam cracking of C2-C4 hydrocarbons, as described in US-A-2011/0040133, US-A-2007/0135668, U.S. Pat. No. 7,964,762, U.S. Pat. No. 6,407,301, US-A-2010/0191031 and US-A-2006/0205988. This results in a mixture of ethene, ethane, acetylene, propene, and additional side products, partially oxygen containing, which have to be separated from the product stream. Acetylene has to be removed as a side product by hydrogenation to ethene. The broad product distribution requires a further processing of the products, such as the metathesis of the olefins.

The preparation of propene by dehydrogenation of propane using a chromium aluminum oxide catalyst is described in U.S. Pat. No. 8,013,201, wherein propene and hydrogen are selectively formed. However, the heat which is required for the dehydrogenation reaction is supplied by burning fossil hydrocarbons such as naphtha or liquid gas corrupting the energy balance as well as the CO₂ balance of this process.

EP-B-0832056 (DE-T2-69520661) describes the dehydrogenation of alkanes using dehydrogenation catalysts, which contain reducible metal oxides such as oxides of Bi, In, Sb, Zn, Tl, Pb or Te and additional metals such as Cr, Mo, Ga or Zn.

In summary, the following problems in the field of transforming C2-C4 alkanes to olefins arise from the prior art:

During the catalytic reaction of C2-C4-alkanes in the presence of Zeolites, the produced olefins further react to give aromatic or polyaromatic compounds (coke), due to the presence of Brønsted-acidic centers. High temperatures of more than 800° C. also promote the formation of coke by further reactions of alkyl aromatic compounds, which contain radicals. The formation of coke blocks the catalyst and thereby deactivates it. Therefore, it is necessary in these cases to continuously regenerate the catalyst by burning off the formed coke. This results in a loss of carbon and idle times in the industrial reaction of C2-C4-alkanes to olefins. Furthermore, given the high process temperatures, substantial costs of material have to be expected for plant construction.

During thermal cracking of alkanes in the presence of steam, which proceeds via radicals, a mixture of ethene, ethane, acetylene, propene, allene and additional side products, partially oxygen containing, results, which necessitates a major separation effort.

The separation of ethene or propene from a C2-C4-alkane/olefin mixture requires a multi-stage distillation, which involves a high expenditure of energy. For an effective separation of ethene and propene from a C2-C4-alkane/olefin mixture, olefin selective membranes have been developed. For example, U.S. Pat. No. 6,878,409 describes silver salt containing polymembranes for the separation of olefins from an olefin/paraffin mixture. U.S. Pat. No. 7,250,545 describes polyimide membranes for the separation of olefins from an olefin/paraffin mixture. U.S. Pat. No. 7,361,800 discloses chitosan based membranes. Finally, U.S. Pat. No. 7,491,262 discloses a silver nanoparticle containing polymembrane, wherein the silver particles are distributed in the nanomatrix. The use of such olefin selective membranes represents a more effective method for the separation of olefins from an olefin/alkane mixture than the separation by distillation. A prerequisite for the applicability of such selective membranes is, however, that the alkane/olefin mixture is free of acetylene, therefore making the suppression of the formation of acetylene desirable.

The problem of the present invention is the provision of a more inexpensive and more environmentally sound process for the preparation of olefins, which does not form undesired side products.

According to the present invention, this problem is solved by the use of a catalyst, which comprises the following components a) and b):
a) at least one metal compound selected from a group consisting of metal carbide, -nitride, -silicide, -phosphide and -sulfide or mixtures thereof, wherein the metal is selected from a group consisting of molybdenum, tungsten, tantalum, vanadium, titanium, niobium, lanthanum and chromium; and
b) at least one non-Brønsted-acidic binder selected from a group consisting of AlPO₄, Bentonite, AlN and N₄Si₃.

The catalyst, used according to the present invention, can be used in a process and in a device for the preparation of olefins. During said process, no undesired side products are formed and said process can be conducted inexpensively and ecologically sound.

The catalyst, used according to the present invention, provides the advantage that it works without Brønsted-acidic components and below a temperature of 800° C., thereby preventing the formation of aromatic compounds, gasoline and polyaromatic compounds (coke). Thus, an energy intensive separation of said undesired side products is not required. In particular, it is advantageous that the formation of coke is prevented; therefore, a removal of said coke and a thereby caused shutdown of the plant are not necessary. The process according to the present invention and the device for the execution of the process according to the present invention can be operated continuously, the energy for the removal of said coke is saved, the CO₂ balance of said process according to the present invention is improved, and a frequent regeneration of the catalyst is not necessary.

Additionally, it is advantageous that by using a reduced reaction temperature of less than 800° C. for the reaction of C2-, C3- or C4-alkanes or a mixture thereof in the presence of the catalyst, used according to the present invention, the formation of acetylene as a side product is suppressed, which simplifies the separation process of the product mixture and makes it more efficient and economic, since a hydrogenation of acetylene to ethene is omitted and selective membranes for the separation of olefins and not dehydrated alkanes can be used, which further improves the energy balance of the process according to the present invention.

A further advantage to be mentioned is that no steam has to be added during the process according to the present invention, thereby preventing the formation of oxygenates and other oxygen containing secondary products and significantly simplifying the separation of the olefins from the product mixture. Furthermore, the energy for heating and separating the steam is saved.

The term “alkanes”, as used in the context of the present invention, refers to saturated, acyclic organic compounds of the general formula CₙH₂ₙ₊₂.

The terms “C2-alkane”, “C3-alkane” and “C4-alkane” as used in a context of the present invention, refer to saturated, acyclic organic compounds of the general formula CₙH₂ₙ₊₂ with n=2, 3 and 4. Accordingly, the C2-alkane refers to C₂H₆ (ethane), the C3-alkane refers to C₃H₈ (propane) and the C4-alkane refers to C₄H₁₀ (linear isomer=n-butane; branched isomer=iso-butane).

The term “olefin”, as used in the context of the present invention, refers to unsaturated, acyclic organic compounds of the general formula CₙH₂ₙ. C₂H₄ describes ethene, C₃H₆ refers to propene, C₄H₈ refers to butene, which comprises the isomers n-butene (1-butene), cis-2-butene, trans-2-butene and iso-butene (2-methyl-1-propene). The term “olefin” is used equivalently with the term “alkene”.

The term “dehydrogenation”, as used in a context of the present invention, refers to the oxidation of alkanes to olefins under the release of hydrogen. The present invention relates to the dehydrogenation of C2-, C3-, or C4-alkanes or a mixture thereof under the formation of olefins in the presence of the catalyst according to the present invention. Accordingly, in the context of the present invention, the term “dehydrogenation” is used synonymously with the term catalytic dehydrogenation. An intermediate of the catalytic dehydrogenation can be a transition metal mediated alpha and/or beta-hydride-elimination.

The term “cracking”, as used in the context of the present invention, refers to the catalytic cleavage of hydrocarbons in the presence of hydrogen to hydrocarbons of lesser molecular weight. In the context of the present invention, the term “cracking” refers to the catalytic cleavage of C2-, C3- or C4-alkanes to C1-, C2- and C3-alkanes and C2- and C3-alkenes mediated by transition metals. The term “cracking”, as used in the context of the present invention, does not comprise “steam cracking”, which occurs thermally via radicals in the presence of steam. Furthermore, the term “cracking”, as used in the context of the present invention, does not comprise the cracking mediated by zeolites, which results from super acidic Brønsted acid groups of the zeolite and proceeds via carbenium ion intermediates.

The term “Brønsted-acidic”, as used in the context of the present invention, is based on the Brønsted-acid-base-concept, wherein an acid is a proton donator, i.e. releases protons, and a base is a proton acceptor, i.e. receives protons. A Brønsted-acid, which is dissolved in water, lowers the pH value of water below 7. A non-Brønsted-acid, i.e. a chemical compound, which is not Brønsted acidic, does not lower the pH value of water below 7, if dissolved in water.

The term “gas-gas-heat exchange”, as used in a context of the present invention, refers to a process, during which the heat of a hot gas or a gas mixture is used for the heating of a cooler gas or gas mixture. The hot gas is cooled by means of a gas-gas-heat exchange with the cooler gas.

The term “fuel cell”, as used in the context of the present invention, refers to a hydrogen-oxygen (or air) fuel cell, wherein hydrogen reacts as fuel with oxygen as an oxidation agent under formation of electric energy and heat energy. The fuel cell consists of a cathode and an anode, which are separated by an electrolyte. Hydrogen is oxidized at the anode and oxygen is reduced at the cathode forming water and energy in an exothermal reaction. A special fuel cell is the solid oxygen fuel cell (SOFC), which is a high temperature fuel cell that is operated at temperatures of 650 to 1000° C. The electrolyte of this cell type consists of a solid ceramic material, which is able to conduct oxygen ions, which is, however, insulating for electrons. Generally, yttrium stabilized zirconium dioxide (YSZ) is used for this purpose. The cathode is also made of ceramic material (e.g. strontium doped lanthanum manganate), which conducts ions and electrons. The anode is made of, e.g. nickel with yttrium doped zirconium dioxide (so-called cermet), which also conducts ions and electrons.

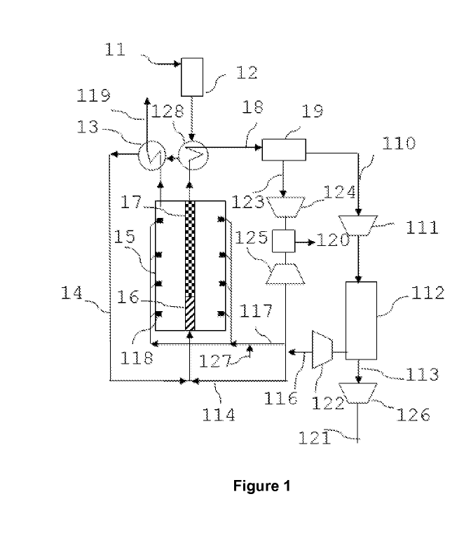

FIG. 1 displays an embodiment of the process according to the present invention and the device according to the present invention for the preparation of olefins from C2-, C3- and C4-alkanes or a mixture thereof, wherein no fuel cell is comprised.

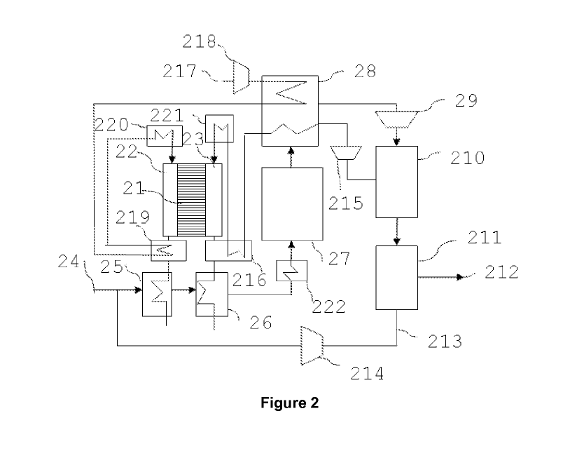

FIG. 2 displays an alternative embodiment of the process according to the present invention and the device according to the present invention for the preparation of olefins from C2-, C3- and C4-alkanes or a mixture thereof, wherein a fuel cell is comprised.

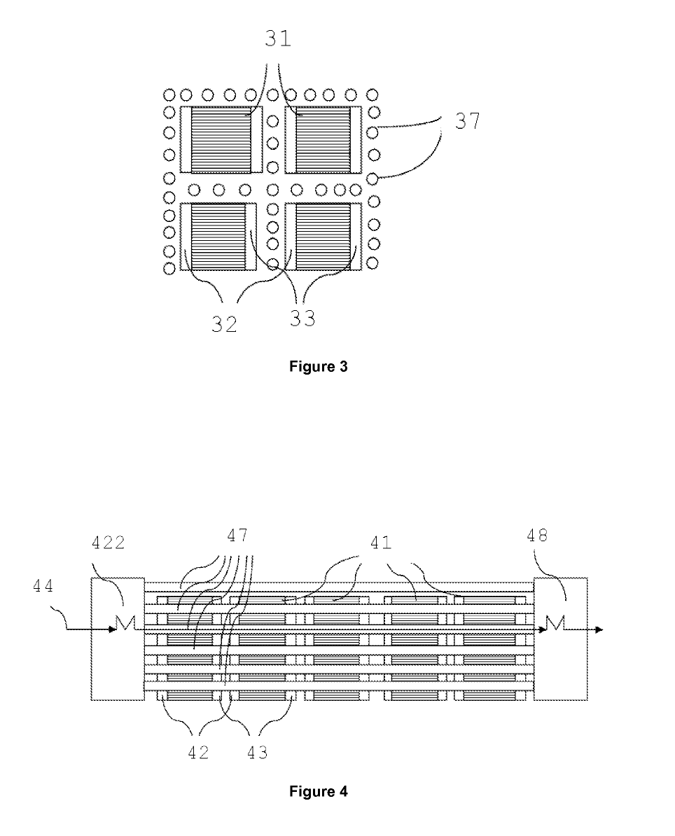

FIG. 3 displays the front view of a tube bundle reactor with reaction tubes running horizontally, which are located in between and above the cell stacks of a SOFC.

FIG. 4 displays the side view of a tube bundle reactor with reaction tubes running horizontally, which are located in between and above the cell stacks of a SOFC.

In the following, the invention will be described in more detail.

### 1. Catalyst

The catalyst used according to the present invention is characterized in that it comprises
a) at least one metal compound selected from a group consisting of metal carbide, -nitride, -silicide, -phosphide and -sulfide or mixtures thereof, wherein the metal is selected from a group consisting of molybdenum, tungsten, tantalum, vanadium, titanium, niobium, lanthanum and chromium, and
b) at least one non-Brønsted-acidic binder selected from a group consisting of AlPO₄, Bentonite, AlN and N₄Si₃.

In a preferred embodiment, which can be combined with any of the previous and subsequent embodiments, the catalyst used according to the present invention comprises 20-95% (w/w) of the metal compound a), based on the total weight of the catalyst. In especially preferred embodiments, the catalyst used according to the present invention comprises 40-95% (w/w), 50-95% (w/w), 60-95% (w/w), 60-90% (w/w) or 60-85% (w/w) of the metal compound a), each based on the total weight of the catalyst. Particularly preferred is a catalyst comprising 60-80% (w/w) of the metal compound a), based on the total weight of the catalyst.

The non-Brønsted-acidic binder, which is selected from a group consisting of AlPO₄, Bentonite, AlN and N₄Si₃, contributes to the catalyst used according to the present invention being porous, thus enlarging its surface. The binder also serves the formation of a coherent agglomerate, thereby conferring on the catalyst a thermally resistant and mechanically resilient geometry. Since the binder is non-Brønsted-acidic, product selectivity is favored towards the formation of olefins; undesired side reactions such as dimerization, formation of aromatic compounds and polymerization are suppressed. In a preferred embodiment of the invention, which can be combined with any of the previous and subsequent embodiments, the non-Brønsted-acidic binder is selected from the group consisting of AlPO₄ and Bentonite. Particularly preferred is AlPO₄ as the non-Brønsted-acidic binder

In a preferred embodiment, which can be combined with any of the previous and subsequent embodiments, the catalyst used according to the present invention comprises 5-80% (w/w) of the non-Brønsted-acidic binder, based on the total weight of the catalyst. In especially preferred embodiments, the catalyst used according to the present invention comprises 5-60% (w/w), 5-50% (w/w), 5-40% (w/w), 10-40% (w/w) or 15-40% (w/w) of the non-Brønsted-acidic binder, each based on the overall amount of the catalyst. Particularly preferred is a catalyst comprising 20-40% (w/w) of the non-Brønsted-acidic binder, based on the total weight of the catalyst.

In a preferred embodiment of the catalyst used according to the present invention which can be combined with any of the previous or subsequent embodiments, the metal compound a) selected from a group consisting of MoₓC, MoₓN, MoₓP, MoₓSi, MoₓS, WₓC, WₓN, WₓP, WₓSi, WₓS, TiₓC, TiₓN, TiₓP, TiₓSi, TiₓS, TaₓC, TaₓN, TaₓP, TaₓSi₂, TaₓSi, TaₓS, VₓC, VₓN, VₓP, VₓSi, VₓS, LaₓC, LaₓN, LaₓP, LaₓSi, LaₓS, NbₓC, NbₓN, NbₓP, NbₓSi, NbₓS, CrₓC, CrₓN, CrₓP, CrₓSi and CrₓS, with 0.1<x<2. In an especially preferred embodiment, which can be combined with any of the previous or subsequent embodiments, 0.2<x≦1. Particularly preferred is 0.5<x<1.

Especially preferred is an embodiment of the catalyst used according to the present invention, which can be combined with any of the previous or subsequent embodiments, wherein the metal compound a) comprises molybdenum, tungsten, niobium, titanium and/or tantalum, and is selected from a group consisting of MoₓC, MoₓN, MoₓP, MoₓSi, MoₓS, WₓC, WₓN, WₓP, WₓSi, WₓS, NbₓC, NbₓN, NbₓP, NbₓSi, NbₓS, TiₓC, TiₓN, TiₓP, TiₓSi, TiₓS, TaₓC, TaₓN, TaₓP, TaₓSi, TaₓSi₂ and TaₓS, with 0.1<x<2, preferred 0.2<x≦1 and particularly preferred 0.5<x<1.

More preferred is an embodiment of the catalyst used according to the present invention, which can be combined with any of the previous or subsequent embodiments, wherein the metal compound a) comprises molybdenum, tungsten, niobium and/or tantalum, and is selected from a group consisting of MoₓC, MoₓN, MoₓP, MoₓSi, MoₓS, WₓC, WₓN, WₓP, WₓSi, WₓS, NbₓC, NbₓN, NbₓP, NbₓSi, NbₓS, TaₓC, TaₓN, TaₓP, TaₓSi, TaₓSi₂ and TaₓS, with 0.1<x<2, preferred 0.2<x≦1 and particularly preferred 0.5<x<1.

More preferred is an embodiment, which can be combined with any of the previous and subsequent embodiments, wherein the catalyst comprises a carbide or nitride or phosphide or silicide of molybdenum, tungsten, tantalum and/or niobium, wherein the component a) is accordingly selected from a group consisting of MoₓC, MoₓN, MoₓP, MoₓSi, WₓC, WₓN, WₓP, WₓSi, TaₓC, TaₓN, TaₓP, TaₓSi, TaₓSi₂, NbₓC, NbₓN, NbₓP and NbₓSi, with 0.1<x<2, preferred 0.2<x≦1 and particularly preferred 0.5<x<1.

More preferred is an embodiment, which can be combined with any of the previous and subsequent embodiments, wherein the catalyst comprises a carbide or nitride or phosphide or silicide of molybdenum, tungsten and/or tantalum, wherein the component a) is accordingly selected from a group consisting of MoₓC, MoₓN, MoₓP, MoₓSi, WₓC, WₓN, WₓP, WₓSi, TaₓC, TaₓN, TaₓP, TaₓSi and TaₓSi₂ with 0.1<x<2, preferred 0.2<x≦1 and particularly preferred 0.5<x<1.

Especially preferred is an embodiment, which can be combined with any of the previous or subsequent embodiments, wherein the component a) comprises a carbide or nitride or phosphide or silicide of tantalum and/or tungsten and thus contains WₓC, WₓN, WₓP, WₓSi, TaₓC, TaₓN, TaₓP, TaₓSi and TaₓSi₂. Particularly preferred are the carbides of tantalum and tungsten as component a), TaₓC and/or WₓC, with 0.1<x<2, preferred 0.2<x≦1 and particularly preferred 0.5<x<1.

In a preferred embodiment of the invention, which can be combined with any of the previous and subsequent embodiments, the catalyst used according to the invention is characterized in that it comprises
a) at least one metal compound selected from a group consisting of metal carbide, -nitride, -silicide, -phosphide and -sulfide or mixtures thereof, wherein the metal is selected from a group consisting of molybdenum, tungsten, tantalum, vanadium, titanium, niobium and lanthanum, and
b) 5-40% (w/w) of a non-Brønsted-acidic binder selected from a group consisting of AlPO₄ and Bentonite.

In a more preferred embodiment, the catalyst used according to the invention is characterized in that component a) is at least one metal compound of the group consisting of MoₓC, MoₓN, MoₓP, MoₓSi, MoₓS, WₓC, WₓN, WₓP, WₓSi, WₓS, TiₓC, TiₓN, TiₓP, TiₓSi, TiₓS, TaₓC, TaₓN, TaₓP, TaₓSi₂, TaₓSi, TaₓS, VₓC, VₓN, VₓP, VₓSi, VₓS, LaₓC, LaₓN, LaₓP, LaₓSi, LaₓS, NbₓC, NbₓN, NbₓP, NbₓSi and NbₓS, wherein 0.1<x<2.0, and in that it comprises 5-40% (w/w) of a non-Brønsted-acidic binder selected from a group consisting of AlPO₄ and Bentonite.

In an even more preferred embodiment, the catalyst used according to the invention is characterized in that the component a) is at least one metal carbide MₓC, metal phosphide MₓP, metal nitride MₓN or metal silicide MₓSi, wherein M stands for a metal which is selected from a group consisting of W, Ta, Nb and Mo, wherein 0.2<x≦1.0, and in that it comprises 5-40% (w/w) of a non-Brønsted-acidic binder selected from a group consisting of AlPO₄ and Bentonite.

The catalyst used according to the present invention may further comprise a carrier material with a large surface. In a preferred embodiment of the invention, which can be combined with any of the previous and subsequent embodiments, the catalyst used according to the present invention comprises at least one non-Brønsted-acidic carrier material selected from a group consisting of TiO₂, Al₂O₃, activated charcoal, SiO₂, SiC and ZrO₂. Especially preferred carrier materials are SiO₂ and SiC and particularly preferred is SiC, due to its heat conductivity of more than 5 Wm⁻¹K⁻¹.

Since the carrier material is preferably non-Brønsted-acidic, the product selectivity is favored toward the formation of olefins and undesired side reactions such as dimerization, the formation of aromatic compounds and polymerization are suppressed.

The catalyst used according to the present invention may contain additional metallic components for the optimization of the catalyst for the catalytic dehydrogenation and cracking of C2-, C3- or C4-alkanes or a mixture thereof.

For the optimization of the catalytic dehydrogenation reaction of C2-, C3- or C4-alkanes or a mixture thereof, the catalyst used according to the present invention comprises in a preferred embodiment, which can be combined with any of the previous and subsequent embodiments, at least one additional metal or a compound containing said metal, wherein the metal is selected from a group consisting of Sn, Ag, Pb, Bi, Mn und Au. In an especially preferred embodiment, which can be combined with any of the previous and subsequent embodiments, the at least one metal is selected from a group consisting of Pb, Ag und Bi. Particularly preferred is Bi.

For the optimization of the catalytic cracking of propane and/or butane, the catalyst used according to the present invention comprises, in a alternative preferred embodiment, which can be combined with any of the previous and subsequent embodiments, at least one additional metal or a compound containing said metal, wherein the metal is selected from a group consisting of Mg, Zn, Ti, Y, La, Sc, V, Al und Cr. In an especially preferred alternative embodiment, which can be combined with any of the previous and subsequent embodiments, the at least one metal is selected from a group consisting of Mg, Sc, Y und La. Particularly preferred is La.

In a preferred embodiment, which can be combined with any of the previous and subsequent embodiments, the at least one metal or a compound thereof is added in an amount of 0.01-10% (w/w) based on the total weight of the catalyst. Especially preferred is an amount of 0.05-5% (w/w) and particularly preferred is an amount of 0.1-1% (w/w).

In a preferred embodiment of the invention, which can be combined with any of the previous and subsequent embodiments, the catalyst displays a bimodule pore geometry which comprises a mixture of mesopores and macropores. In a preferred embodiment of the catalyst used according to the present invention, which can be combined with any of the previous and subsequent embodiments, the mesopores have a size of 0.1-50 nm and the macropores have a size of 50-3000 nm. Especially preferred is a biomodule pore structure with a mixture of mesopores with a size of 2-50 nm and macropores with a size of 50-1500 nm. The pore volume is 0.1-1 cm³/g, preferably 0.12-0.9 cm³/g and particularly preferred 0.2-0.8 cm³/g. The determination of pore size and -volume is carried out according to DIN66133.

In a preferred embodiment of the catalyst, which can be combined with any of the previous and subsequent embodiments, the grain size of the components a) and b) of the catalyst used according to the present invention is 2-3000 nm. Especially preferred is a grain size of 5-800 nm and particularly preferred is a grain size of 10-500 nm. Grain size is determined by laser diffraction according to ISO 13320.

In a preferred embodiment of the catalyst as used according to the invention, which may be combined with any of the previous and subsequent embodiments, the thermal conductivity of metal compound a) is more than 5 Wm⁻¹K⁻¹. More preferred is a thermal conductivity of metal compound a) of 15 Wm⁻¹K⁻¹ and particularly preferred is a thermal conductivity of more than 20 Wm⁻¹K⁻¹, each for a particle size of ≦1 μm of metal compound a).

The surface of metal compound a), determined in accordance with BET-method, is in a preferred embodiment, which can be combined with any of the previous and subsequent embodiments, 0.1-400 m²/g. Particularly preferred is a surface of metal compound a) of 2-390 m²/g.

### 2. Preparation of the Catalyst Used According to the Present Invention

The process for the preparation of the catalyst used according to the present invention comprises the mixing of at least one metal compound a), selected from a group consisting of metal carbide, -nitride, -silicide, -phosphide, and -sulfide or mixtures thereof, wherein the metal is selected from a group consisting of molybdenum, tungsten, tantalum, vanadium, titanium, niobium, lanthanum and chromium, with b), at least one non-Brønsted-acidic binder selected from a group consisting of AlPO₄, Bentonite, AlN and N₄Si₃.

Component a) can be prepared using processes known to the skilled person or can be purchased (e.g. from Treibacher AG or Wolfram AG). The component b) can be also prepared by common processes or can be purchased (e.g. from Sigma Aldrich or Alfa Aesar).

Both components a) and b) are preferably used in the form of powders, which has a grain size of less than 400 nm, preferably less than 150 nm and more preferably less than 50 nm.

The mixing of components a) and b) can be accomplished by using a known mixer, for example, a ribbon mixer, a conical mixer or a Henschel mixer.

In a preferred embodiment of the process for the preparation of the catalyst used according to the present invention additional components are added to the mixture of component a) and b), such as at least one of the above named non-Brønsted-acidic carrier materials and/or at least one of the above named additional metals or compounds containing said metals.

As macropore forming agents can be added soot particles, carbon nanotubes, urea formaldehyde resin, CaCO₃, alkylsilicones, polydiallyldimethylammonium chloride, polystyrene beads, polyvinyl butyral, naphthalene, polyethylene oxide, polypropylene oxide or saw dust. Preferred are pore forming agents which form channels, such as carbon nanotubes or linear polymers such as polyvinyl butyral or linear polycondensation products. The macropore forming agent can be added to the mixture at 2-70% (w/w), preferably 5-65% (w/w) and, in particular, 10-55% (w/w), in each case based on the amount of component a).

A mixture is formed which comprises at least the components a) and b) as powders. In a preferred embodiment of the process for the preparation of the catalyst used according to the present invention the mixture comprises 5-60% (w/w), 5-50% (w/w), 5-40% (w/w), 10-40% (w/w) and 15-40% (w/w) of the non-Brønsted-acidic binder, each based on the total weight of the mixture. Particularly preferred is a mixture comprising 20-40% (w/w) of the non-Brønsted-acidic binder, based on the total weight of the mixture.

In the following the resulting mixture is kneaded with a common Z-blade kneading machine. To accomplish thickening, the kneading is preferably carried out under addition of a liquid in which a pasting agent and/or mesopore forming agent is dissolved in an amount of 0.1-15% (w/w), preferably 0.2-10% (w/w), based on the weight of the liquid. This solution is added in an amount of 1-40% (w/w), preferably 2-20% (w/w), based on the amount of metal component a) and non-Brønsted-acidic binder to the mixture. Oxygenates such as C1-, C2-, C3- or C4-alcohols or water can serve as liquids. As mesopore forming agents can serve hydrophilic polymers such as hydroxy cellulose, polyethylene glycol, alkylated cellulose derivates, starch, cyano ethylated starch, carboxymethylated starch, carboxymethyl cellulose, methyl cellulose, hydroxyethyl cellulose, polyvinyl alcohol, vinyl ether-maleic acid mixed polymers, sodium alginate, sodium lignin sulfonate, gum arabic, gum tragacanth, ammonium alginate, polyvinyl pyrrolidone, citric acid, polyisobutene, polymethacrylate, polyacrylate and polytetrahydrofuran. Primarily, these substances foster the formation of a malleable mass during the kneading process and the subsequently described shaping and drying steps by bridging the primary particles and furthermore insure the mechanical stability of the shaped body during the shaping process and during drying. The substances are subsequently removed from the shaped body by calcination and thus, leave mesopores in the catalyst.

In a preferred embodiment of the process for the preparation of a catalyst used according to the present invention the kneading process is conducted for 5-120 min. Especially preferred are 15-80 min and particularly preferred are 35-60 min.

Subsequently, the resulting mass obtained by the steps described above, at least comprising the components a) and b), is shaped by a shaping process such as tabletting, pelleting or extrusion. The preferred shaping process in the context of the process for the preparation of the catalyst used according to the present invention is the extrusion.

In a preferred embodiment, which can be combined with any of the previous and subsequent embodiments, the shaped mixture is dried at 20-90° C. Especially preferred for the drying is 30-80° C. and particularly preferred is 40-70° C. In a preferred embodiment, which can be combined with any of the previous and subsequent embodiments, the drying period is 0.1-40 h. Especially preferred is a drying period of 1-35 h and particularly preferred is a drying period of 5-30 h.

By means of the shaping process and the drying of the mixture, comprising the components a) and b), a catalyst shape is obtained, which preferably has a diameter of 2-30 mm. Especially preferred is a diameter of 3-25 mm and particularly preferred is a diameter of 4-20 mm.

The shape can take on different geometries, for example, full cylinders with 3-6 axial ridges, hollow cylinders with 1-8 axial holes with a diameter of 2-10 mm, as well as saddles with U or Y geometry. To insure an improved axial temperature distribution and a reduced loss of pressure, the geometry of a preferred embodiment of the catalyst used according to the present invention, which can be combined with any of the previous and subsequent embodiments, is a cylinder with 3 to 7 axial holes with a diameter of 2-4 mm and/or a cylinder with 4-6 axial ridges.

In an alternative preferred embodiment, which can be combined with any of the previous and subsequent embodiments, the catalyst used according to the present invention can be designed as a monolith with honeycomb structure. The monolith has a preferred outer cylinder geometry with a diameter of 30-150 mm and a length of 15-5000 mm. The honeycomb structure of the monolith catalyst is characterized by continuous parallel open channels and possesses 1-55 holes/cm², preferably 2-50 holes/cm² and more preferably 4-40 holes/cm². The channels can exhibit round, rectangular or triangular geometry. The diameters of the parallel channels are 1-4 mm and the wall thicknesses are 0.05-2 mm.

In a preferred embodiment of the process for the preparation of the catalyst used according to the present invention, which can be combined with any of the previous and subsequent embodiments, a calcination step (calcination) under He, N₂, Ar, CH₄, H₂, or C2-, C3-, C4-alkane atmosphere or a mixture of said gases follows the drying step for tempering of the catalyst.

The calcination can be conducted in a common rotating calcination kiln or a shaft or tube furnace. In a preferred embodiment, the temperature of the calcination is 500-700° C., preferably 530-680° C. In a preferred embodiment, the calcination time is 30 min −10 h. In a preferred embodiment, the heating rate during calcination is 1-10° C./min, more preferably 1-5° C./min. If the calcination is conducted in the presence of an inert gas such as N₂, Ar or He, in a preferred embodiment of the process for the preparation of the catalyst used according to the present invention, a reduction step follows the calcination. The reduction step is conducted in the presence of C2-, C3- or C4-alkanes. During said reduction, remaining oxygen containing compounds are reduced.

### 3. Use of the Catalyst According to the Present Invention

The catalyst according to the invention can be used for combined steam free, catalytic cracking and dehydrogenation of C2-, C3- and C4-alkanes or a mixture thereof.

In particular, the catalyst used according to the present invention is suited for catalytic dehydrogenation of C2- and C3-alkanes (ethane and propane) and for steam free catalytic cracking of C3- and C4-alkanes (propane and butane).

The catalyst used according to the present invention can be used for the preparation of olefins from C2-, C3- or C4-alkanes or a mixture thereof by catalytic dehydrogenation and cracking. In particular, the catalyst used according to the present invention is suited for the preparation of ethene and propene from C2-, C3- or C4-alkanes or a mixture thereof. In particular, the catalyst used according to the present invention is suited for the preparation of ethene from C2-, C3- or C4-alkanes or a mixture thereof.

In particular, ethene can be prepared by the reaction of ethane and propane and butane or a mixture thereof with the catalyst used according to the present invention. In particular, propene can be prepared by the reaction of propane and butane or a mixture thereof with the catalyst used according to the present invention. The selectivity for the formation of ethene from propane and butane can be favored by raising the reaction temperature.

The catalyst used according to the present invention can also be used in the subsequently described process for the preparation of olefins from C2-, C3- or C4-alkanes or a mixture thereof. The catalyst used according to the present invention can also be used in the context of the subsequently described device for the preparation of olefins from C2-, C3- or C4-alkanes or a mixture thereof.

### 4. Process Using the Catalyst According to the Present Invention

The catalyst used according to the present invention is preferably used in a process for the preparation of olefins. A preferred process according to the present invention for the preparation of olefins from C2-, C3- or C4-alkanes or a mixture thereof comprises the following steps:
a) Heating of a C2-, C3- or C4-alkane or a mixture thereof,
b) Passing the heated C2-, C3- or C4-alkane or the mixture thereof over a catalyst, wherein the catalyst comprises
   i) at least one metal compound selected from a group consisting of metal carbide, -nitride, -silicide, -phosphide, and -sulfide or mixtures thereof, wherein the metal is selected from a group consisting of molybdenum, tungsten, tantalum, vanadium, titanium, niobium, lanthanum and chromium, and
   ii) at least one non-Brønsted-acidic binder selected from a group consisting of AlPO₄, Bentonite, AlN and N₄Si₃,
   whereby a product mixture is formed comprising at least one olefin, methane and hydrogen; and
c) Separation of the product mixture.

In the context of the process according to the present invention, the above described catalyst according to the present invention is used, said catalyst comprising at least one metal compound selected from a group consisting of metal carbide, -nitride, -silicide, -phosphide and -sulfide or mixtures thereof, wherein the metal is selected from a group consisting of molybdenum, tungsten, tantalum, vanadium, titanium, niobium, lanthanum and chromium and at least one non-Brønsted-acidic binder selected from a group consisting of AlPO₄, Bentonite, AlN and N₄Si₃.

In a particularly preferred embodiment, which can be combined with any of the previous or subsequent embodiments, the process according to the present invention for the preparation of olefins from C2-, C3- or C4-alkanes or a mixture thereof comprises
a) Heating of a C2-, C3- or C4-alkane or a mixture thereof,
b) Passing the heated C2-, C3- or C4-alkane or the mixture thereof over a catalyst, wherein the catalyst comprises
   i) at least one metal compound selected from a group consisting of metal carbide, -nitride, -silicide, -phosphide, and -sulfide or mixtures thereof, wherein the metal is selected from a group consisting of molybdenum, tungsten, tantalum, vanadium, titanium, niobium and lanthanum, and
   ii) at least one non-Brønsted-acidic binder selected from a group consisting of AlPO₄, and Bentonite,
   whereby a product mixture is formed comprising at least one olefin, methane and hydrogen; and
c) Separation of the product mixture.

The additional preferred embodiments of the catalyst used in the context of the process according to the present invention are identical with the preferred embodiments of the above described catalyst according to the present invention.

C2-, C3- or C4-alkanes or a mixture thereof serve as starting materials (educts, starting alkanes, reactants) for the preparation of olefins according to the present invention. The term “starting materials” is used according to its common meaning in the art and thus refers to the constitution of the C2-, C3- and C4-alkanes or a mixture thereof before and during the passing over the catalyst used according to the present invention. During the passing over the catalyst used according to the present invention, the starting materials are consumed and transformed into the reaction products, which form the product mixture, as subsequently clarified.

In a preferred embodiment of the process according to the present invention, which can be combined with any of the previous and subsequent embodiments, the C2-, C3- and C4-alkanes or a mixture thereof are completely drained and desulfurized before heating and/or passing over the catalyst used according to the present invention. Said draining and desulfurization is carried out under atmospheric pressure or elevated pressure by means of industrially applicable absorbents for water and sulfur containing products known to the skilled person, such as molecular sieve 5 Å.

In a preferred embodiment of the process according to the present invention, which can be combined with any of the previous and subsequent embodiments, up to 50% (v/v), based on the volume of the C2-, C3- or C4-alkanes or a mixture thereof, of at least one additional gas selected from a group consisting of CH₄ and N₂ and/or H₂ is added to the C2-, C3- or C4-alkanes or a mixture thereof before heating and/or passing over the catalyst used according to the present invention. A preferred gas for this purpose is CH₄. The addition of a further gas reduces the partial pressure of the starting materials, which improves the turnover of the catalytic reaction. At the same time, increased ethene product selectivity is achieved by suppression of side reactions of the C2-, C3- and C4-alkanes or a mixture thereof during heating.

For catalytic reaction with the catalyst used according to the present invention, in the context of the process according to the present invention, the C2-, C3- or C4-alkanes or a mixture thereof are brought to the necessary reaction temperature, required for the reaction, by heating. Said heating comprises preheating of the C2-, C3- or C4-alkanes or a mixture thereof.

The preheating of the cool C2-, C3- or C4-alkanes or a mixture thereof is preferably carried out by gas-gas-heat exchange with a hot gas or gas mixture in a gas-gas-heat exchanger. The term “cool” means that the compounds have room temperature (20-30° C.) before heating. The hot product mixture, which results from the catalytic reaction of the C2-, C3- or C4-alkanes or a mixture thereof with the catalyst used according to the present invention can be used as the hot gas or gas mixture for the preheating by heat exchange. Alternatively or in addition, the hot gases of a gas burner can also be used for preheating the C2-, C3- and C4-alkanes by means of gas-gas-heat exchange. Alternatively, also the hot anode exhaust gas or cathode exhaust gas of a fuel cell can be used for preheating of the C2-, C3- and C4-alkanes or a mixture thereof by means of gas-gas-heat exchange.

In addition to preheating by gas-gas-heat exchange, the C2-, C3- or C4-alkanes or a mixture thereof can also be preheated by an electrical or gas powered heating element.

In a preferred embodiment of the process according to the present invention, which can be combined with any of the previous or subsequent embodiments, the C2-, C3- or C4-alkanes or a mixture thereof are preheated to temperatures of less than 900° C., preferably less than 800° C. Especially preferred are temperatures of 400-700° C., of 500-750° C. and in particular temperatures of 600-790° C.

The C2-, C3- and C4-alkanes or a mixture thereof preheated according to the above described process are heated to reaction temperature for the catalytic reaction with the catalyst used according to the present invention in the next step, which happens preferably in the preheating zone of a reactor and wherein either the heat of a gas burner or the heat generated in a fuel cell is used.

Also, the catalyst is preferably heated to reaction temperature by the heat of a gas burner or the heat generated in a fuel cell.

In a preferred embodiment of the process according to the present invention, which can be combined with any of the previous and subsequent embodiments, the C2-, C3- or C4-alkanes or a mixture thereof and the catalyst are heated to a reaction temperature of less than 900° C., preferably less than 800° C. Especially preferred are temperatures of 400-790° C., 480-780° C., 550-770° C. and 600-760° C. Particularly preferred are temperatures of 670-750° C.

The C2-, C3- or C4-alkanes heated to reaction temperature or a mixture thereof is subsequently passed over the catalyst used according to the present invention. In a preferred embodiment of the process according to the present invention, which can be combined with any of the previous and subsequent embodiments, the cracking and dehydrogenation, catalyzed by the catalyst used according to the present invention, occurs at a temperature of less than 1100° C., preferably less than 900° C., particularly preferably less than 800° C. Preferably, the cracking and dehydrogenation with the catalyst used according to the present invention occurs at a temperature of 400-790° C., 500-780° C. and 600-770° C. Particularly preferred is a temperature of 670-760° C. For the combined catalytic cracking and dehydrogenation of propane and butane, temperatures of less than 700° C. are preferred and especially preferred are temperatures of 600-690° C. For the reaction of ethane, temperatures of 700-790° C. are preferred.

The dwelling time of the starting compounds over the catalyst used according to the present invention is defined by the “gas hourly space velocity” (GHSV), which defines the volume of the starting materials in relation to the volume of the catalyst bed. In a preferred embodiment, which can be combined with any of the previous and subsequent embodiments, the GHSV is 10-50,000 h⁻¹. Especially preferred is a GHSV of 20-45,000 h⁻¹. Particularly preferred is a GHSV of 30-35,000 h⁻¹.

In a preferred embodiment of the process according to the present invention, which can be combined with any of the previous and subsequent embodiments, the pressure during the catalytic reaction of the C2-, C3- or C4-alkanes or a mixture thereof is 0.1-20 bar. Especially preferred is a pressure of 0.2-10 bar and 0.3-6 bar. Particularly preferred is a pressure of 0.5-5 bar.

During the passing over the catalyst used according to the present invention, the heated C2-, C3- or C4-alkanes or a mixture thereof are cracked or dehydrated. Thereby a product mixture is formed from the starting materials, which comprises at least one olefin, methane and hydrogen. The at least one olefin is ethene, propene or butene or a mixture thereof. Preferably, the at least one olefin is mixture of ethene and propene with preferably a higher amount of ethene in the mixture. Additionally, further cracking products can be present in the product mixture, such as C2- and C3-alkanes.

In a preferred embodiment of the process according to the present invention, the hot product mixture is cooled after the catalytic reaction to prevent a further reaction of the reaction products with each other. The cooling is preferably conducted by gas-gas-heat exchange with the cool starting materials for the catalytic reaction, which can happen for example in a gas-gas-heat exchanger. Alternatively, the cooling medium for the gas-gas-heat exchange with the hot product mixture can be cool air or an N₂/O₂-mixture and/or hydrogen, which, as described in the following, is separated from the product mixture and may be cooled by a sequence of compression and decompression after being separated.

In the next step of the process according to the present invention, the product mixture is separated, i.e. the product mixture is separated in its individual components (olefins, non-dehydrated alkanes and hydrogen resulting from cracking) by the subsequently described separation method. During said separation method, either methane and hydrogen are first separated from the product mixture and subsequently the remaining olefin/alkane mixture is separated or first hydrogen is separated from the product mixture and then the remaining alkane/olefin mixture is separated.

In a preferred embodiment of the process according to the present invention, which can be combined with any of the previous and subsequent embodiments, methane resulting from cracking and hydrogen resulting as a side product from dehydrogenation is removed from the product mixture. This is preferably accomplished by an industrially applicable low temperature distillation unit (demethanizer), which is known to the skilled person. In this unit, a compressor is coupled with a turbo expander. The product mixture is initially compressed to approximately 80 bar and subsequently cooled to as little as −120° C. by decompression to 20 bar. Thereby the C2-, C3- and C4-components of the product mixture are liquefied while methane and hydrogen remain gaseous and can be separated as a methane/hydrogen mixture.

In a preferred embodiment, which can be combined with any of the previous and subsequent embodiments, the methane/hydrogen mixture separated from the product mixture is used as fuel for a gas burner for heating the starting materials and the catalyst.

Alternatively, the hydrogen can be separated from the separated methane/hydrogen mixture by means of an industrially applicable hydrogen selective absorption process, which is known to the skilled person, for example, by means of a hydrogen selective membrane, thus obtaining a hydrogen fraction and a methane fraction. The hydrogen selective membrane can be, for example, a zeolite with small pores (SAPO-34), which is loaded with silanes, Cu or Ag, or palladium metal membranes, carbon molecular sieves or carbon nanotubes. The separation of hydrogen from the methane/hydrogen mixture by means of a selective membrane is preferably carried out at a temperature of 25-200° C. and a pressure of 5-50 bar.

The methane fraction obtained after separation of the hydrogen can be used as fuel for a gas burner for heating the C2-, C3- and C4-alkanes or a mixture thereof or a catalyst used according to the present invention. The methane fraction can also be added to the starting materials for dilution before the catalytic reaction of said starting materials with the catalyst used according to the present invention. In a preferred embodiment of the process according to the present invention, which can be combined with any of the previous and subsequent embodiments, the methane fraction is divided and used as fuel for a gas burner for the heating of the C2-, C3- and C4-alkanes or a mixture thereof and the catalyst according to the present invention as well as added to the starting materials for dilution before their catalytic reaction.

In an alternative preferred embodiment, which can be combined with any of the previous and subsequent embodiments, hydrogen alone is initially separated from the product mixture, which can be accomplished by an industrially applicable hydrogen selective absorption process, which is known to the skilled person, for example, by means of a hydrogen selective membrane. The separated hydrogen may be cooled by a sequence of compression and decompression steps and subsequently used for the cooling of the hot product mixture by means of gas-gas-heat exchange in a gas-gas-heat exchanger.

After separation of hydrogen or of hydrogen and methane from the product mixture, the separation of the remaining olefin/alkane mixture comprising the at least one olefin formed by catalytic cracking/dehydrogenation according to the present invention from C2- and C3-alkane cracking products.

In a preferred embodiment of the process according to the present invention, which can be combined with any of the previous and subsequent embodiments, the separation of the remaining olefin/alkane mixture occurs by industrially applicable separation processes, which are known to the skilled person. These commonly known separation processes comprise selective adsorption processes, multi-stage distillation processes, separation by means of olefin selective membranes or the liquefaction of the olefins by means of decompression and cooling.

In an especially preferred embodiment of the process, which can be combined with any of the previous and subsequent embodiments, the separation of the remaining olefin/alkane mixture occurs at a temperature of 25-80° C. and a pressure of 5-30 bar by means of at least one olefin selective membrane, which separates the at least one formed olefin from the non-dehydrated alkane cracking products. Said olefin/alkane separation in the olefine selective membrane occurs via 7-interactions with Ag⁺, Cu⁺ or Fe⁺ ions or binding to Ag, Cu or Fe nanoparticles. Said ions or particles can be deposited on SiO₂ or integrated in a polymer matrix.

Said especially preferred embodiment for the separation of the desired olefin reaction products from the non-dehydrated alkanes by means of a selective membrane works in the context of the present invention because during the reaction of the C2-, C3- or C4-alkanes or a mixture thereof with the catalyst according to the present invention no acetylene is formed as a side product due to the low working temperature. Thus the energy expenditure and procedural effort for the separation steps of said process is minimized.

If a mixture of olefins, consisting of ethene, propene and/or butene, is obtained after separation of the remaining olefin/alkane mixture in the olefin-selective membrane, for the further separation of the olefin reaction products ethene, propene and butene from each other, distillation separation processes, which are known to the skilled person, can be used. The olefins thus isolated can be compressed by means of a compressor and stored.

In a preferred embodiment of the process according to the present invention, which can be combined with any of the previous and subsequent embodiments, the alkane cracking products separated from the remaining olefin/alkane mixture are either partially or completely channeled into a gas burner as fuel for the heating of the starting materials and the catalyst and/or added to the starting materials before the reaction according to the present invention.

In a preferred embodiment of the process according to the present invention, which can be combined with any of the previous and subsequent embodiments, the hydrogen isolated as described above is channeled into a fuel cell, which is preferably a high temperature fuel cell and more preferably a solid oxide fuel cell (SOFC), for the use as anode fuel gas, wherein it is electrochemically transformed with air or a O₂/N₂ mixture to water under generation of heat and electricity. The heating of the hydrogen to an anode inlet temperature of 700-800° C. can be accomplished in the context of the process according to the present invention by gas-gas-heat exchange with the hot product mixture of the catalytic reaction with the catalyst used according to the present invention. Additionally, the heat of the cathode reaction of the fuel cell as well as electrical heaters or gas burners can be used for the heating of the hydrogen. In a preferred embodiment of the process according to the present invention, the heating of the air or O₂/N₂ mixture, comprising preferably more than 20% O₂ (v/v) in relation to the total mixture of the O₂/N₂ mixture, to the cathode inlet temperature is achieved by gas-gas-heat exchange with the hot product mixture of the catalytic reaction according to the present invention. Additionally, the heat of the cathode reaction as well as electrical heaters or gas burners can be used for the heating of the air or the O₂/N₂ mixture.

The electricity generated in the fuel cell can be used for the operation of electrical heaters which can be optionally used, as described above, for the pre-heating of the starting materials. The hot cathode and anode exhaust gases produced in the fuel cell can be used for the pre-heating of the starting materials by gas-gas-heat exchange. The heat generated in the fuel cell can be used as an alternative to the heating with a gas burner for the heating of the catalyst used according to the present invention.

### 5. Device Using the Catalyst According to the Present Invention

The catalyst according to the present invention is preferably used in a device for the preparation of olefins. A preferred device according to the present invention for the preparation of olefins from C2-, C3- or C4-alkanes or a mixture thereof comprises:
a) at least one heating unit for pre-heating of a C2-, C3- and C4-alkane or a mixture thereof,
b) at least one reactor comprising a heating element and a catalyst, wherein the catalyst comprises:
   (i) at least one metal compound selected from a group consisting of metal carbide, -nitride, -silicide, -phosphide and -sulfide or mixtures thereof, wherein the metal is selected from a group consisting of molybdenum, tungsten, tantalum, vanadium, titanium, niobium, lanthanum and chromium, and
   (ii) at least one non-Brønsted-acidic binder selected from a group consisting of AlPO₄, Bentonite, AlN and N₄Si₃;
c) at least two separation units for the separation of gases.

Thereby, the above described catalyst according to the present invention, comprising at least one metal compound selected from a group consisting of metal carbide, -nitride, -silicide, -phosphide and -sulfide or mixtures thereof, wherein the metal is selected from a group consisting of molybdenum, tungsten, tantalum, vanadium, titanium, niobium, lanthanum and chromium, and at least one non-Brønsted-acidic binder selected from a group consisting of AlPO₄, Bentonite, AlN and N₄Si₃, which is also used in the context of the process according to the present invention, is used in the context of the device according to the present invention.

In a particularly preferred embodiment, which can be combined with any of the previous or subsequent embodiments, the device according to the present invention for the preparation of olefins from C2-, C3- or C4-alkanes or a mixture thereof comprises
a) at least one heating unit for pre-heating of a C2-, C3- and C4-alkane or a mixture thereof,
b) at least one reactor comprising a heating element and a catalyst, wherein the catalyst comprises:
   (i) at least one metal compound selected from a group consisting of metal carbide, -nitride, -silicide, -phosphide and -sulfide or mixtures thereof, wherein the metal is selected from a group consisting of molybdenum, tungsten, tantalum, vanadium, titanium, niobium and lanthanum, and
   (ii) at least one non-Brønsted-acidic binder selected from a group consisting of AlPO₄ and Bentonite;
c) at least two separation units for the separation of gases.

The further preferred embodiments of the catalyst used in the context of the device according to the present invention are identical with the preferred embodiments of the catalyst according to the present invention described above, which is also used in the context of the process according to the present invention. In the context of the device according to the present invention, the catalyst is a component of a reactor in which the catalytic reaction occurs. Besides the catalyst used according to the present invention, the reactor of the device according to the present invention comprises a heating element. Preferably, the reactor additionally comprises a pre-heating zone, wherein the pre-heated C2-, C3- or C4-alkanes or a mixture thereof are brought to the final reaction temperature by the heating element of the reactor. Preferably, the catalyst is contained in a reaction zone of the reactor.

The reactor can be designed as a tube reactor or as a plate reactor. Alternatively, it can be designed in a V-shaped form or in other geometry.

In a preferred embodiment of the device according to the present invention, which can be combined with any of the previous and subsequent embodiments, the reactor is a tube reactor, for example, a fixed bed tube reactor or a tube bundle reactor. Particularly preferred is a tube bundle reactor.

In a tube reactor, the reaction zone with the catalyst used according to the present invention is located in a reaction tube or in a bundle of reaction tubes as a fixed bed. Preferred tube inner diameters for this purpose are 2.5-20 cm, especially preferred 2.6-15 cm and particularly preferred 2.7-10 cm. The length of the tubes is 5-50 m, preferably 7-35 m, more preferably 9-30 m. For a better heat transfer, the reaction tube or the bundle of reaction tubes can be equipped on their exterior with elongated lamellae or spiraled ridges.

Preferably, a pre-heating zone precedes the reaction zone with the catalyst used according to the present invention. Said pre-heating zone can be a fixed bed of inert ceramics, for example SiC, with high heat conductivity. Also, the catalyst used according to the present invention itself can serve as a pre-heating fixed bed due to its heat connectivity.

In a preferred embodiment of the device according to the present invention the heating element refers to at least one gas burner, for example, a stage burner or a radiant wall burner.

Particularly preferred is a lateral radiant wall burner. Said lateral radiant wall burner can be designed as a ceramic burner.

The gas burner is used for the heating of the pre-heating zone as well as the reaction zone with the catalyst used according to the present invention. In a tube bundle reactor, the reaction tubes are heated indirectly by burning a gas, for example hydrogen or methane, in the space that surrounds the reaction tubes. The fuel gases can be obtained from the product mixture as described above and channeled into the burner together with air. The exhaust gases of the gas burner can also be used for pre-heating of the C2-, C3- and C4-alkanes or a mixture thereof.

In an alternative preferred embodiment of the device according to the present invention, the heating element is a fuel cell, wherein the heat generated by electrochemical reaction of hydrogen with oxygen is used for heating the pre-heating and reaction zone.

In an especially preferred embodiment of the device according to the present invention, which can be combined with any of the previous and subsequent embodiments, the reactor is a tube bundle reactor comprising a SOFC as heating element. The tube bundles of the reactor run horizontally and are located in between and above the SOFC cell stacks, thereby providing an efficient heat transfer from the SOFC to the tube bundles of a reactor.

The heating element of the reactor heats the pre-heating and reaction zone within the reactor to temperatures of preferably less than 1100° C., especially preferably less than 900° C., 400-790° C., 500-780° C. and 600-770° C. Particularly preferred is a temperature of 670-760° C.

The working pressure in the reaction zone is preferably within a range of 0.1-20 bar. Especially preferred is a pressure of 0.2-10 bar and 0.3-6 bar. Particularly preferred is a pressure of 0.5-5 bar.

Before the C2-, C3- and C4-alkanes or a mixture thereof are passed over the catalyst according to the present invention, they are heated to reaction temperature said heating comprising a pre-heating of the C2-, C3- or C4-alkanes or a mixture thereof as described in the context of the process according to the present invention. Therefore, the device according to the present invention comprises at least one heating unit for pre-heating of the C2-, C3- or C4-alkanes or a mixture thereof.

In a preferred embodiment of the device according to the present invention, which can be combined with any of the previous and subsequent embodiments, the at least one heating unit for pre-heating of the C2-, C3- or C4-alkanes or a mixture thereof refers to at least one gas-gas-heat exchanger, wherein the pre-heating of the C2-, C3- or C4-alkanes or a mixture thereof is accomplished by heat exchange with a hot gas or a hot gas mixture. Preferably the hot product mixture of the catalytic reaction is used for the heat transfer in said at least one gas-gas-heat exchanger. Alternatively or additionally the hot exhaust gases of a gas burner can be used for the gas-gas-heat exchange with the starting materials of the catalytic reaction in the present of the catalyst used according to the present invention. Therefore, in a especially preferred embodiment, which can be combined with any of the previous and subsequent embodiments, the device according to the present invention comprises at least two gas-gas-heat exchangers as heating units for the preheating of the starting materials, wherein the starting materials are preheated in one gas-gas-heat exchanger with the hot product mixture and additionally said starting materials are preheated in the other gas-gas-heat exchanger by the hot exhaust gases of a gas burner. The gas burner required for said purpose can also be heating element of the reactor of the device according to the present invention.

In alternative preferred embodiment, wherein the reactor comprises a fuel cell as heating element, the preheating of the starting materials occurs in the at least one gas-gas-heat exchanger by heat exchange with the hot cathode and anode exhaust gases of the fuel cell reaction. In an especially preferred embodiment, which can be combined with any of the previous and subsequent embodiments, the device according to the present invention comprises two gas-gas-heat exchangers as heating units for preheating the starting materials, wherein the starting materials are preheated in one gas-gas-heat exchanger with the hot cathode exhaust gases and additionally in the other gas-gas-heat exchanger with the hot anode exhaust gases of the fuel cell.

In a preferred embodiment, which can be combined with any of the previous and subsequent embodiments, the device according to the present invention comprises in addition to the at least one heat exchanger at least one supplementary heater for the preheating of the starting materials. Said supplementary heater can be a gas burner or a electrical heater. In an especially preferred embodiment of the device according to the present invention, which can be combined with any of the previous and subsequent embodiments, at least two supplementary heaters are comprised and particularly preferred are three supplementary heaters for the preheating of the C2-, C3- and C4-alkanes or a mixture thereof.

In preferred embodiment, which can be combined with any of the previous and subsequent embodiments, the device according to the present invention comprises at least one absorber for draining and desulfurizing the C2-, C3- and C4-alkanes or a mixture thereof before their heating and reaction by means of the catalyst according to the present invention. The absorber contains industrially applicable absorbent materials for water and sulfur-containing products, which are known to the skilled person, such as 5 Å molecular sieves.

In a preferred embodiment, which can be combined with any of the previous and subsequent embodiments, the device according to the present invention comprises at least one cooling unit for the cooling of the hot product mixture subsequent to the catalytic reaction with the catalyst used according to the present invention. At least one gas-gas-heat exchanger is especially preferred as the at least one cooling unit, wherein the cooling of the hot product mixture occurs with a cooler gas or gas mixture. The cooling of the hot reaction mixture by heat exchange in a gas-gas-heat exchanger with the cool starting materials before the heating of said starting materials is preferred. Alternatively or additionally air or a N₂/O₂ mixture with more than 20% (v/v) oxygen can be used for the cooling of the hot reaction products in the gas-gas-heat exchanger as well as hydrogen cooled by compression and decompression, which was regained from the product mixture.

For the separation of the product mixture, the device according to the present invention comprises at least two separation units. The at least two separation units refer to gas separation units, which are able to separate the product mixture, which comprises at least one olefin, methane and hydrogen into its individual components.

In a preferred embodiment of the device according to the present invention, which can be combined with any of the previous and subsequent embodiments, the at least two separation units of the device according to the present invention comprise at least one industrially applicable low temperature distillation unit (demethanizer), which is known to the skilled person, for the separation of methane and hydrogen from the product mixture, as well as at least one industrially applicable olefin/paraffin gas separation unit, which is known to the skilled person, for the separation of the remaining olefin/alkane mixture. Said low temperature distillation unit is placed after the reactor and before the olefin/paraffin gas separation unit. For the further separation of the methane/hydrogen mixture obtained by the low temperature distillation unit the device according to the present invention may comprise an additional separation unit, preferably a hydrogen selective membrane, for the separation of the hydrogen from the methane.

In an alternative preferred embodiment, which can be combined with any of the previous and subsequent embodiments, the at least two separation units of the device according to the present invention comprise at least one industrially applicable separation unit for the separation of hydrogen, which is known to the skilled person, as well as at least one industrially applicable olefin/paraffin gas separation unit for the separation of the remaining olefin/alkane mixture, which is known to the skilled person. Said separation of hydrogen can be accomplished by a hydrogen selective membrane or by other absorption processes, which are known to the skilled person. A hydrogen selective membrane for the separation of the hydrogen from the product mixture is preferred. The separation unit for the hydrogen is preferably placed after the reactor and before the before the olefin/paraffin gas separation unit. The separated hydrogen can be used as anode fuel gas for a fuel cell.

The olefin/paraffin gas separation unit can be a multistage distillation device, one or multiple selective membranes or a unit for the liquefaction of the olefins by means of decompression and cooling. In a especially preferred embodiment of the device according to the present invention, which can be combined with any of the previous and subsequent embodiments, the separation of the olefins from the alkanes occurs in the olefin/paraffin gas separation unit by means of industrially applicable selective membranes, which are known to the skilled person, which separate the at least one olefin from the non-dehydrated alkane cracking products.

The hydrogen isolated from the product mixture may serve as anode fuel gas for the fuel cell comprised in a preferred embodiment of the device according to the present invention. For heating of the fuel gas hydrogen to an anode inlet temperature of 700-800° C. and for heating of the air or N₂/O₂ mixture the device according to the present invention comprises preferably at least one gas-gas-heat exchanger, wherein the hydrogen is heated by the hot product mixture of the catalytic reaction and additionally by the heat of the cathode reaction. Furthermore at least one electrical heater or gas burner may be comprised for the heating of the anode fuel hydrogen.

In the preferred embodiment of the device according to the present invention comprising a fuel cell, the endothermic dehydrogenation reaction under formation of hydrogen and olefins is combined with the exothermic reaction of hydrogen with oxygen or air in the fuel cell. The process according to the present invention is provided with the resulting electricity and the resulting heat for the heating of the starting materials as described above.

FIG. 1 displays an embodiment of the process according to the present invention or the device according to the present invention for the preparation of olefins from C2-, C3- or C4-alkanes or a mixture thereof, wherein no fuel cell is comprised. This embodiment can be combined with any other embodiment of the present invention. Displayed is a reactor 15 with lateral gas burner 118, a preheating zone 16 and a reactor chamber with the catalyst used according to the present invention 17.

The starting materials 11 consisting of C2-, C3- and C4-alkanes or a mixture thereof are initially drained and desulfurized by means of a 5 Å molecular sieve in the absorber 12.

Subsequently the starting materials 11 are preheated to a temperature of less than 800° C. via the gas-gas-heat exchanger 13 and the preheating zone 16. For preheating the hot reaction products 18 after reaction in the presence of the catalyst used according to the present invention and the hot exhaust gases 119 of the lateral gas burner 118 are used.

After preheating of the starting materials 11 by the hot reaction products 18 and the hot exhaust gases 119, the starting materials are brought to a reaction temperature of 600-790° C. in the preheating zone 16 of the reactor, which is heated by lateral gas burner 118.

Subsequently the starting materials are channeled into the reactor chamber 17, in which the catalyst according to the invention for the reaction of the C2-, C3- and C4-alkanes or a mixture thereof is located.

After the catalytic reaction with the catalyst used according to the present invention, the product mixture 18 is cooled with the gas-gas-heat exchanger 128 and subsequently channeled into the low temperature distillation unit 19, wherein the separation of methane and hydrogen from the remaining olefin/alkane mixture occurs.

After the separation of methane and hydrogen in the low temperature distillation unit 19 the olefin/alkane mixture 110 is compressed by means of the compressor 111 and separated in the olefin/paraffin gas separation unit 112 by means of at least one olefin selective membrane.

After separation of the olefins 113, said olefins are compressed in the compressor 126 before their further use.

The methane and hydrogen 123 separated by the low temperature distillation unit 19 may be compressed by the compressor 124. If required, hydrogen 120 may be separated from the stream of methane.

The stream of methane 117 may be completely or partially channeled into the burner 118 or as a dilution of up to 50% added to the starting materials 14 via line 114 before or after the heat exchanger 13. The hydrogen/methane mixture 123 after the low temperature distillation unit, the pure methane 117 or a mixture of 123 and the alkane cracking products 116 is used as fuel.

The alkane cracking products 116 may be channeled in total to the burner 118 after decompression 122 or further added to the starting materials before of after the heat/exchanger 13. The separation unit for the separation of hydrogen 120 is a hydrogen selective membrane known to the skilled person.

FIG. 2 displays an alternative embodiment of the process according to the present invention or the device according to the present invention for the preparation of olefins from C2-, C3- or C4-alkanes or a mixture thereof, wherein the hydrogen separated from the product mixture is channeled into a industrially applicable high temperature fuel cell 21 known to the skilled person. The heat resulting from the electrochemical reaction of hydrogen with oxygen in said high temperature fuel cell is used in this embodiment for the preheating of the starting materials and for the heating of the reactor, wherein the catalyst used according to the present invention is located. Furthermore, electricity, which may be used for operating electrical heaters, which in turn may be used for preheating of the starting materials, is generated in the fuel cell.

In this alternative embodiment of the process according to the present invention or the device according to the present invention the endothermic catalytic dehydrogenation reaction for the preparation of olefins from C2-, C3- and C4-alkanes or a mixture thereof during which hydrogen is released is thus combined with the exothermic electrochemical reaction of the hydrogen with air or a O₂/N₂ mixture in a fuel cell under formation of heat and electricity. The process according to the present invention can thus be provided with the generated heat energy and electrical energy.

FIG. 2 displays a high temperature fuel cell 21 with an oxygen or air containing cathode 22 and a fuel gas containing anode 23.

The starting materials 24 consisting of C2-, C3- and C4-alkanes or a mixture thereof are desulfurized before the catalytic reaction and are preheated to a temperature of less than 800° C. by the gas-gas-heat exchanger 25 and 26, optionally by an additional heater 222, which can be an electrical heater or a gas burner.

Subsequently, the reaction products are channeled into the reactor 27, in which the catalyst used according to the present invention is located.

The product mixture is cooled via the gas-gas-heat exchanger 28. Air or a N₂/O₂ mixture with more than 20% (v/v) oxygen 217 after a slight compression 218 as well as the hydrogen, which is separated in the separation unit 210 after the compression 29 and largely decompressed in unit 215 is used as cooling medium.

The separation unit 210 is an industrially applicable hydrogen selective membrane selectively passing hydrogen, which is known to the skilled person.

The slightly compressed air or N₂/O₂ mixture 217 is heated to the required inlet temperature by the additional heat exchanger 219 and 220.

The cool hydrogen, after the decompression, serves a quick cooling of the product mixture in the gas-gas-heat exchanger 28 to suppress a further reaction of the reaction products and thereby elevating the olefin product selectivity. The hydrogen is further heated by the cathode exhaust gases in the gas-gas-heat exchanger 216 after heating in the gas-gas-heat exchanger 28 and maybe additionally heated to the required anode inlet temperature between 700-800° C. by a third heater 221, which can be electrical heater or a burner.

The remaining olefin/alkane mixture is separated into olefins and alkanes in the paraffin/olefin separation unit 211. The alkanes are channeled back into the process after decompression 214.

The paraffin/olefin separation unit 211 is an industrially applicable separation unit known to the skilled person, which contains at least one olefin selective membrane.

It is noted that the preheating of the starting materials is not restricted to the sequence of the heat exchangers and may also be carried out initially via the gas-gas-heat exchanger 28 and subsequently via the additional heater 222 (electrical heater or a gas burner). With this heat management the preheating of the hydrogen separated in the separation unit 210 is carried out after the decompression 215 via the heat exchanger 26, 216 and additionally via the heater 221 (electrical heater or a gas burner). The air or a O₂/N₂ mixture 217 is heated after the slight compression 218 via the heat exchangers 25, 219 and additionally via the heater 220 (electrical heater or a gas burner).

The process according to the present invention and the device according to the present invention using the catalyst according to the present invention are suitable for the preparation of olefins from C2-, C3- and C4-alkanes or a mixture thereof. In particular the process and the device are suitable for the preparation of ethene from ethane and/or propane and/or butane or for the preparation of propene from propane and/or butane. The product selectivity for the formation of ethene from the C2-, C3- and C4-alkanes or a mixture thereof can be regulated by elevating the temperature.

The process according to the present invention may be carried out in stepwise fashion with two serially connected reactors. For this purpose, in the first step propane and butane are heated to less than 700° C. and cracked catalytically by means of the catalyst according to the present invention. The product mixture is separated by means of an olefin selective membrane into olefins and alkanes. The cracking product ethane is catalytically dehydrated to ethene in a second reactor at 750 to less than 800° C., said ethene is isolated from the product mixture by an olefin selective membrane.

FIG. 3 displays the preferred embodiment of a tube bundle reactor, which is coupled with the cell stacks of a SOFC 31 (frontal view). In said tube bundle reactor the reaction tubes 37 of said reactor run horizontally and are located in between and above the cell stacks of the SOFC 31. Thereby an efficient heat transfer from the SOFC to the tube bundles is ensured. Furthermore, the cathodes and anodes 32 and 33 of the fuel cell stacks can be seen.

FIG. 4 displays the preferred embodiment of a tube bundle reactor, which is coupled with the cell stacks 41 of a SOFC (side view). In said tube bundle reactor the reaction tubes 47 of said reactor run horizontally and are located in between and above the cell stacks 41 of the SOFC. Furthermore, the cathodes and anodes 42 and 43 of the fuel cell stacks as well as the heat exchangers 48 and 422 can be seen and the starting materials 44 consisting of C2-, C3- or C4-alkanes or a mixture thereof.

The process according to the present invention provides the advantage that the olefin/alkane comprising product mixture can be separated into alkanes and olefins by olefin selective membranes. Thereby the alkanes can be added again to the process either for diluting the starting materials or as fuel gas in a gas burner for the heating of the starting materials to reaction temperature. This affords an improved CO₂-balance as well as an improved heat management and thereby an improved energy balance.

The embodiment displayed in FIG. 2 provides the advantage that by the combination of the exothermic electrochemical reaction in the fuel cell with the endothermic dehydrogenation reaction ethene and/or propene and electricity and heat can be produced. The electricity produced in this manner in turn may be used for the heating of the starting materials, for example, by means of electrically operated heaters, as displayed in FIG. 2, which renders the process energy efficient and lowers the CO₂ emissions of the process according to the present invention. The heat produced in said manner can be used for the heating of the starting materials and the catalyst as depicted in FIGS. 2, 3 and 4. Also in this embodiment an improved CO₂ balance as well as an improved heat management and therefore an improved energy balance is achieved.

In the following the invention will be described in more detail by means of examples. However, the invention is not limited by these examples.

### EXAMPLES

### Example 1

### Preparation of a WC/AlPO₄ Catalyst

80 g WC powder (Wolfram AG) with a grain size of 450 nm is mixed with 20 g AlPO₄ as a non-Brønsted-acidic binder (Alfa Aesar), to result in a WC/AlPO₄ mixture with 20% (w/w) AlPO₄, based on the total weight. To this mixture 4 ml aqueous starch solution 8% (w/w) is added while the mixture is further mixed and kneaded for 60 min by means of a kneader. The resulting mixture is pressed to tablets of 4 mm diameter and 3 mm thickness and dried for 5 hours at 50° C. In the subsequent calcination step one heats with 2° C./min to 570° C. while passing N₂ over the mixture and maintains the temperature for 3 hours at 570° C. Subsequently the calcined catalyst is brought to reaction temperature with 5° C./min in a tube reactor in the presence of H₂ or C2-, C3- or C4-alkanes and reduced for at least 1 hour.

### Example 2

### Preparation of a MoC/AlPO₄ Catalyst

80 g MoC powder (Treibacher AG) with a grain size of 450 nm is mixed with 20 g AlPO₄ (Alfa Aesar) as a non-Brønsted-acidic binder, to result in a MoC/AlPO₄ mixture with 20% (w/w) AlPO₄, based on the total weight. To this mixture 4 ml aqueous starch solution 8% (w/w) is added while the mixture is further mixed and kneaded for 60 min by means of a kneader. The resulting mixture is pressed to tablets of 4 mm diameter und 3 mm thickness and dried for 5 hours at 50° C. In the subsequent calcination step one heats with 2° C./min to 570° C. while passing N₂ over the mixture and maintains the temperature for 3 hours at 570° C. Subsequently the calcined catalyst is brought to reaction temperature with 5° C./min in a tube reactor in the presence of H₂ or C2-, C3- or C4-alkanes and reduced for at least 1 hour.

### Example 3

### Preparation of a TiC/AlPO₄ Catalyst

50 g TiC powder (Alfa Aesar) is mixed 15 g AlPO₄ (Alfa Aesar) as a non-Brønsted-acidic binder, to result in a TiC/AlPO₄ mixture with 23% (w/w) AlPO₄, based on the total weight. To this mixture 11 ml aqueous starch solution 8% (w/w) is added while the mixture is further mixed and kneaded for 60 min by means of a kneader. The resulting mixture is pressed to tablets of 4 mm diameter und 3 mm thickness and dried for 10 hours at 40° C. In the subsequent calcination step one heats with 2° C./min to 570° C. while passing N₂ over the mixture and maintains the temperature for 3 hours at 570° C. Subsequently the calcined catalyst is brought to reaction temperature with 5° C./min in a tube reactor in the presence of H₂ or C2-, C3- or C4-alkanes and reduced for at least 1 hour.

### Example 4

### Preparation of a TiN/AlPO₄ Catalyst

25 g TiN powder (Alfa Aesar) is mixed 5 g AlPO₄ (Alfa Aesar) as a non-Brønsted-acidic binder, to result in a TiN/AlPO₄ mixture with 17% (w/w) AlPO₄, based on the total weight. To this mixture 5 ml aqueous starch solution 8% (w/w) is added while the mixture is further mixed and kneaded for 60 min by means of a kneader. The resulting mixture is pressed to tablets of 4 mm diameter und 3 mm thickness and dried for 10 hours at 40° C. In the subsequent calcination step one heats with 2° C./min to 570° C. while passing N₂ over the mixture and maintains the temperature for 3 hours at 570° C. Subsequently the calcined catalyst is brought to reaction temperature with 5° C./min in a tube reactor in the presence of H₂ or C2-, C3- or C4-alkanes and reduced for at least 1 hour.

### Example 5

### Preparation of a TaC/AlPO₄ Catalyst

30 g TaC powder (Alfa Aesar) is mixed 6 g AlPO₄ (Alfa Aesar) as a non-Brønsted-acidic binder, to result in a TaC/AlPO₄ mixture with 17% (w/w) AlPO₄, based on the total weight. To this mixture 7 ml aqueous starch solution 8% (w/w) is added while the mixture is further mixed for 60 min and kneaded by means of a kneader. The resulting mixture is pressed to tablets of 4 mm diameter und 3 mm thickness and dried for 12 hours at 30° C. In the subsequent calcination step one heats with 2° C./min to 570° C. while passing N₂ over the mixture and maintains the temperature for 3 hours at 570° C. Subsequently the calcined catalyst is brought to reaction temperature with 5° C./min in a tube reactor in the presence of H₂ or C2-, C3- or C4-alkanes and reduced for at least 1 hour.

### Example 6

### Preparation of a TaN/AlPO₄ Catalyst

20 g TaN powder (Alfa Aesar) is mixed 4 g AlPO₄ (Alfa Aesar) as a non-Brønsted-acidic binder, to result in a TaN/AlPO₄ mixture with 17% (w/w) AlPO₄, based on the total weight. To this mixture 6 ml aqueous starch solution 8% (w/w) is added while the mixture is further mixed for 60 min and kneaded by means of a kneader. The resulting mixture is pressed to tablets of 4 mm diameter und 3 mm thickness and dried for 12 hours at 30° C. In the subsequent calcination step one heats with 2° C./min to 570° C. while passing N₂ over the mixture and maintains the temperature for 3 hours at 570° C. Subsequently the calcined catalyst is brought to reaction temperature with 5° C./min in a tube reactor in the presence of H₂ or C2-, C3- or C4-alkanes and reduced for at least 1 hour.

### Example 7

### Preparation of a CrC/AlPO₄ Catalyst

30 g CrC powder (Alfa Aesar) is mixed 6 g AlPO₄ (Alfa Aesar) as a non-Brønsted-acidic binder, to result in a CrC/AlPO₄ mixture with 17% (w/w) AlPO₄, based on the total weight. To this mixture 7 ml aqueous starch solution 8% (w/w) is added while the mixture is further mixed for 60 min and kneaded by means of a kneader. The resulting mixture is pressed to tablets of 4 mm diameter und 3 mm thickness and dried for 10 hours at 40° C. In the subsequent calcination step one heats with 2° C./min to 570° C. while passing N₂ over the mixture and maintains the temperature for 3 hours at 570° C. Subsequently the calcined catalyst is brought to reaction temperature with 5° C./min in a tube reactor in the presence of H₂ or C2-, C3- or C4-alkanes and reduced for at least 1 hour.

### Example 8

### Preparation of a NbC/AlPO₄ Catalyst

26 g NbC powder (Alfa Aesar) is mixed 5 g AlPO₄ (Alfa Aesar) as a non-Brønsted-acidic binder, to result in a NbC/AlPO₄ mixture with 16% (w/w) AlPO₄, based on the total weight. To this mixture 5 ml aqueous starch solution 8% (w/w) is added while the mixture is further mixed for 60 min and kneaded by means of a kneader. The resulting mixture is pressed to tablets of 4 mm diameter und 3 mm thickness and dried for 10 hours at 40° C. In the subsequent calcination step one heats with 2° C./min to 570° C. while passing N₂ over the mixture and maintains the temperature for 3 hours at 570° C. Subsequently the calcined catalyst is brought to reaction temperature with 5° C./min in a tube reactor in the presence of H₂ or C2-, C3- or C4-alkanes and reduced for at least 1 hour.

### Example 9

### Preparation of a WC/AlN Catalyst

50 g WC powder (Alfa Aesar) is mixed 11 g AlN (Alfa Aesar) as a non-Brønsted-acidic binder, to result in a WC/AlN mixture with 17% (w/w) AlPO₄, based on the total weight. To this mixture 5 ml aqueous starch solution 8% (w/w) is added while the mixture is further mixed for 60 min and kneaded by means of a kneader. The resulting mixture is pressed to tablets of 4 mm diameter und 3 mm thickness and dried for 12 hours at 30° C. In the subsequent calcination step one heats with 2° C./min to 570° C. while passing N₂ over the mixture and maintains the temperature for 3 hours at 570° C. Subsequently the calcined catalyst is brought to reaction temperature with 5° C./min in a tube reactor in the presence of H₂ or C2-, C3- or C4-alkanes and reduced for at least 1 hour.

### Example 10

### Preparation of Ethene from Ethane by Means of WC/AlPO₄ and MoC/AlPO₄ Catalysts

Ethane is drained and desulfurized in an absorber, which contains a molecular sieve 5 Å. Subsequently, the drained and desulfurized ethane is heated to temperatures below 750° C. The heated ethane is passed over the catalyst (WC/AlPO₄ catalyst of Example 1 or MoC/AlPO₄ catalyst of Example 2) with a GHSV of 60 h⁻¹.

Table 1 displays the product distribution after the reaction of ethane in the presence of the WC/AlPO₄ catalyst of Example 1 at different temperatures. Table 2 shows the product distribution after the reaction of ethane in the presence of the MoC/AlPO₄ catalyst of Example 2 at different temperatures.

TABLE 1
Reaction of ethane to ethene with the WC/AIPO₄
catalyst of Example 1 at different temperatures.

| | Component (%, v/v) | | | |
|---|---|---|---|---|
| | | | C₂H₆ + | |
| | C₂H₆ | C₂H₆ | 25% (v/v) CH₄ | C₂H₆ |
| | Temperature | | | |
| | 690° C. | 700° C. | 740° C. | 740° C. |
| H₂ | 23 | 24.2 | 22.5 | 28.2 |
| CH₄ | 4.1 | 7.8 | 23.9 | 14.6 |
| C₂H₆ | 43.2 | 36.3 | 18.6 | 20.1 |
| C₂H₄ | 26.3 | 29.7 | 35.0 | 32.8 |
| select. C₂H₄ | 26.3 | 29.7 | 35.0 | 32.8 |

TABLE 2
Reaction of ethane to ethene with the MoC/AIPO₄
catalyst of Example 2 at different temperatures.

| | Component (%, v/v) | | | |
|---|---|---|---|---|
| | C₂H₆ | C₂H₆ | C₂H₆ | C₂H₆ + N₂ |
| | Temperature | | | |
| | 700° C. | 710° C. | 730° C. | 740° C. |
| H₂ | 25.7 | 26.1 | 31.7 | 33.5 |
| CH₄ | 7.6 | 7.5 | 11.6 | 10.2 |
| C₂H₆ | 35.2 | 35.7 | 24.6 | 21.8 |
| C₂H₄ | 29.4 | 29.1 | 32 | 34.2 |
| select. C₂H₄ | 29.4 | 29.1 | 32 | 34.2 |

Hence, the best product selectivities for the reaction of ethane to ethene are achieved at a temperature of 740° C. The ethene product selectivity increases by diluting the starting material ethane with CH₄ (Table 1, column 4).

### Example 11

### Preparation of Ethene from Propane or Butane by Means of a WC/AlPO₄ Catalyst

Propane (or butane) is drained and desulfurized in an absorber, which contains a molecular sieve 5 Å. Subsequently, the drained and desulfurized propane (butane) is heated to temperatures of less than 800° C. via a gas-gas-heat exchanger and subsequently passed over the catalyst (WC/AlPO₄ catalyst of Example 1) with a GHSV of 60 h⁻¹.

Table 3 displays the product distribution after the reaction of propane and butane with the WC catalyst of Example 1 at different temperatures.

TABLE 3
Reaction of propane and butane with the WC/AIPO₄ catalyst of
Example 1 at different temperatures.

| | Component (%, v/v) | | | | |
|---|---|---|---|---|---|
| | U.S. Pat. No. | | | | |
| | 4,929,791 | | | | |
| | C₃H₈ | | | C₃H₈ + | |
| | HZSM50 | C₃H₈ | C₃H₈ | 25% (v/v) N₂ | C₄H₁₀ |
| | Temperature | | | | |
| | 650° C. | 670° C. | 696° C. | 696° C. | 650° C. |
| H₂ | 0 | 5 | 0.6 | 0.5 | 0.7 |
| CH₄ | 37.2 | 41 | 44.5 | 44.4 | 31.3 |
| C₂H₆ | 2.9 | 6.5 | 12.5 | 6 | 13.7 |
| C₂H₄ | 27.7 | 39 | 43.3 | 48 | 29.6 |
| C₂H₆ | 11.7 | 8 | 1 | 1 | 24.7 |
| BTX* | 10.5 | 0 | 0 | 0 | 0 |
| select. | 55.4 | 78 | 86.6 | 96 | 59.2 |
| C₂H₄ | | | | | |

*BTX = Benzene, Toluene, Xylene

Table 3 shows that high ethene selectivities are accomplished at 696° C. using propane as starting material. The selectivity can be further improved by dilution of the propane with 25% (v/v) N₂. Furthermore it is illustrated that using the catalyst according to the invention suppresses the formation of aromatic compounds.

### Example 12

### Preparation of Ethene from Ethane and Propane by Means of TiC/AlPO₄ and TiN/AlPO₄ Catalysts

Ethane (or propane) is drained and desulfurized in an absorber, which contains a molecular sieve 5 Å. Subsequently, the drained and desulfurized ethane (propane) is heated to temperatures below 800° C. and is passed over the catalyst.

Table 4 shows the product distribution after reaction of propane and ethane with the TiC/AlPO₄ catalyst of Example 3 and the TiN/AlPO₄ catalyst of Example 4 at different temperatures.

TABLE 4
Reaction of propane and ethane to ethene at different
temperatures and different GHSV values with TiC/AIPO₄ and
TiN/AIPO₄ catalysts from Examples 3 and 4.

| | Component | | | |
|---|---|---|---|---|
| | TiC/AIPO₄ | TiC/AIPO₄ | TiN/AIPO₄ | TiN/AIPO₄ |
| | Feedgas | | | |
| | C₃H₈ | C₂H₆ | C₃H₈ | C₂H₆ |
| | Temperature | | | |
| | 696° C. | 740° C. | 696° C. | 740° C. |
| GHSV (h⁻¹) | 40 | 40 | 40 | 40 |
| H₂ (%, v/v) | 3.1 | 20 | 6 | 19.8 |
| CH₄ (%, v/v) | 41.8 | 16.7 | 38.7 | 15.2 |
| C₂H₆ (%, v/v) | 8.5 | 31.6 | 5.7 | 33.3 |
| C₂H₄ (%, v/v) | 38.2 | 31.8 | 36.8 | 31.7 |
| C₃H₆ (%, v/v) | 11.5 | — | 13.2 | — |
| select. C₂H₄ | 76.4 | 31.8 | 73.5 | 31.7 |

### Example 13

### Preparation of Ethene from Ethane and Propane by Means of TaC/AlPO₄ and TaN/AlPO₄ Catalysts

Ethane (or propane) is drained and desulfurized in an absorber, which contains a molecular sieve 5 Å. Subsequently, the drained and desulfurized ethane (propane) is heated to temperatures below 800° C. and is passed over the catalyst.

Table 5 shows the product distribution after reaction of propane and ethane with the TaC/AlPO₄ catalyst of Example 5 and the TaN/AlPO₄ catalyst of Example 6 at different temperatures.

TABLE 5
Reaction of propane and ethane to ethene at different temperatures and different GHSV
values with the TaC/AlPO₄ and the TaN/AlPO₄ catalyst from the Examples 5 and 6.

| Component | TaN/AlPO₄ | TaN/AlPO₄ | TaN/AlPO₄ | TaC/AlPO₄ | TaC/AlPO₄ | TaC/AlPO₄ |
|---|---|---|---|---|---|---|
| Feedgas | C₃H₈ | C₃H₈ | C₂H₆ | C₃H₈ | C₂H₆ | C₂H₆ |
| Temperature | 696° C. | 696° C. | 740° C. | 696° C. | 740° C. | 740° C. |
| GHSV (h⁻¹) | 80 | 40 | 40 | 80 | 20 | 40 |
| H₂ (%, v/v) | 14.9 | 5.0 | 29.1 | 18 | 8.3 | 25.2 |
| CH₄ (%, v/v) | 14.5 | 43.8 | 10.0 | 15.1 | 39.1 | 13.6 |
| C₂H₆ (%, v/v) | 5.2 | 8.6 | 27.1 | 5.1 | 28.4 | 26.4 |
| C₂H₄ (%, v/v) | 25.8 | 37.1 | 32.6 | 24.6 | 24.2 | 33.6 |
| C₃H₈ (%, v/v) | 22.4 | 1.9 | — | 19.5 | — | — |
| C₃H₆ (%, v/v) | 17.1 | 3.8 | 1.2 | 17.8 | — | 1.4 |
| select. C₂H₄ | 51.7 | 74.2 | 32.6 | 49.2 | 24.2 | 33.6 |

### Example 14

### Preparation of Ethene from Ethane and Propane by Means of CrC/AlPO₄ and NbC/AlPO₄ Catalysts

Ethane (or propane) is drained and desulfurized in an absorber, which contains a molecular sieve 5 Å. Subsequently, the drained and desulfurized ethane (propane) is heated to temperatures below 800° C. and is passed over the catalyst.

Table 6 shows the product distribution after reaction of propane and ethane with the CrC/AlPO₄-catalyst of Example 7 and the NbC/AlPO₄ catalyst of Example 8 at different temperatures.

TABLE 6
Reaction of propane and ethane to ethene at different temperatures and different GHSV
values with the CrC/AlPO₄ and the NbC/AlPO₄ catalyst from the Examples 7 and 8.

| Component | CrC/AlPO₄ | CrC/AlPO₄ | CrC/AlPO₄ | NbC/AlPO₄ | NbC/AlPO₄ | NbC/AlPO₄ |
|---|---|---|---|---|---|---|
| Feedgas | C₃H₈ | C₂H₆ | C₂H₆ | C₃H₈ | C₂H₆ | C₂H₆ |
| Temperature | 696° C. | 740° C. | 740° C. | 696° C. | 696° C. | 740° C. |
| GHSV (h⁻¹) | 40 | 20 | 40 | 80 | 40 | 40 |
| H₂ (%, v/v) | 3 | 2 | 11 | 15.2 | 7.9 | 27 |
| CH₄ (%, v/v) | 42.7 | 48.4 | 36.9 | 14.8 | 39.0 | 13.4 |
| C₂H₆ (%, v/v) | 12.6 | 28.4 | 27 | 5.2 | 3.7 | 28.0 |
| C₂H₄ (%, v/v) | 37.9 | 21.2 | 24.3 | 24.3 | 34.3 | 31.5 |
| C₃H₈ (%, v/v) | 1 | — | — | 21.5 | 4.6 | — |
| C₃H₆ (%, v/v) | 2.9 | — | — | 18.7 | 10.2 | — |
| select. C₂H₄ | 75.8 | 21.2 | 24.3 | 48.6 | 68.5 | 31.5 |

### Example 15

### Preparation of Ethene from Ethane and Propane by Means of a WC/AlN Catalyst

Ethane (or propane) is drained and desulfurized in an absorber, which contains a molecular sieve 5 Å. Subsequently, the drained and desulfurized ethane (propane) is heated to temperatures below 800° C. and is passed over the catalyst.

Table 7 shows the product distribution after reaction of propane and ethane with the WC/AlN catalyst of Example 9.

TABLE 7
Reaction of propane and ethane at different temperatures
and different GHSV values with WC/AIN catalyst from Example 9.

| | Component | | | |
|---|---|---|---|---|
| | WC/ALN | WC/ALN | WC/AIN | WC/ALN |
| | Feedgas | | | |
| | C₃H₈ | C₃H₈ | C₂H₆ | C₂H₆ |
| | Temperature | | | |
| | 695° C. | 695° C. | 740° C. | 740° C. |
| GHSV (h⁻¹) | 80 | 40 | 40 | 30 |
| H₂ (%, v/v) | 14 | 3 | 17 | 10 |
| CH₄ (%, v/v) | 21.9 | 42.7 | 21.4 | 43.6 |
| C₂H₆ (%, v/v) | 8.8 | 12.6 | 29.9 | 22.7 |
| C₂H₄ (%, v/v) | 26.3 | 37.9 | 31.6 | 23.6 |
| C₃H₈ (%, v/v) | 12.3 | 1 | — | — |
| C₃H₆ (%, v/v) | 16.7 | 2.9 | — | — |
| select. C₂H₄ | 52.6 | 75.8 | 31.6 | 23.6 |

1. A catalyst, characterized in that it comprises:
a) at least one metal compound selected from a group consisting of metal carbide, -nitride, -silicide, -phosphide and -sulfide or mixtures thereof, wherein the metal is selected from a group consisting of molybdenum, tungsten, tantalum, vanadium, titanium, niobium, lanthanum and chromium, and
b) 5-40% (w/w) of a non-Brønsted-acidic binder selected from a group consisting of AlPO₄, Bentonite, AlN and N₄Si₃.

2. The catalyst of claim 1, characterized in that the component a) is at least one metal compound of the group consisting of MoₓC, MoₓN, MoₓP, MoₓSi, MoₓS, WₓC, WₓN, WₓP, WₓSi, WₓS, TiₓC, TiₓN, TiₓP, TiₓSi, TiₓS, TaₓC, TaₓN, TaₓP, TaₓSi₂, TaₓSi, TaₓS, VₓC, VₓN, VₓP, VₓSi, VₓS, LaₓC, LaₓN, LaₓP, LaₓSi, LaₓS, NbₓC, NbₓN, NbₓP, NbₓSi and NbₓS, wherein 0.1<x<2.0.

3. The catalyst of claim 1 characterized in that the component a) is at least one metal carbide MₓC, metal phosphide MₓP, metal nitride MₓN or metal silicide MₓSi, wherein M stands for a metal which is selected from a group consisting of W, Ta, Nb and Mo, wherein 0.2<x≦1.0.

4. The catalyst of claim 1 characterized in that it comprises at least one non-Brønsted-acidic carrier material, which is selected from a group consisting of TiO₂, Al₂O₃, activated charcoal, SiO₂, SiC and ZrO₂.

5. A process for the preparation of a catalyst according to claim 1 characterized in that it comprises a mixing of the components a) and b).

6. A process for the preparation of olefins from C2-, C3- or C4-alkanes or a mixture thereof characterized in that it comprises the following steps:
a) heating of a C2-, C3- and C4-alkane or a mixture thereof,
b) passing the heated C2-, C3- or C4-alkanes or the mixture thereof over a catalyst, wherein the catalyst comprises
i) at least one metal compound selected from a group consisting of metal carbide, -nitride, -silicide, -phosphide and -sulfide or mixtures thereof, wherein the metal is selected from a group consisting of molybdenum, tungsten, tantalum, vanadium, titanium, niobium, lanthanum and chromium, and
ii) at least one non-Brønsted-acidic binder selected from a group consisting of AlPO₄, Bentonite, AlN and N₄Si₃;
whereby a product mixture is formed which comprises at least one olefin, methane and hydrogen; and
c) separating the product mixture.

7. The process according to claim 6 characterized in that the temperature in step a) lies between 400° C. and 800° C.

8. The process according to claim 6 characterized in that the at least one olefin is separated from the product mixture by absorption by means of at least one olefin selective membrane.

9. The process according to claim 6 characterized in that the hydrogen is absorbed from the product mixture, channeled into a fuel cell, and transformed in the fuel cell as anode fuel gas with air or an O₂/N₂ mixture under formation of electricity and heat.

10. A device for the preparation of olefins from C2-, C3- or C4-alkanes or a mixture thereof characterized in that it comprises:
a) at least one heating unit for pre-heating of a C2-, C3- and C4-alkane or a mixture thereof,
b) at least one reactor comprising a heating element and a catalyst, wherein the catalyst comprises
i) at least one metal compound selected from a group consisting of metal carbide, -nitride, -silicide, -phosphide and -sulfide or mixtures thereof, wherein the metal is selected from a group consisting of molybdenum, tungsten, tantalum, vanadium, titanium, niobium, lanthanum and chromium, and
ii) at least one non-Brønsted-acidic binder selected from a group consisting of AlPO₄, Bentonite, AlN and N₄Si₃;
c) at least two separation units for the separation of gases.

11. The device according to claim 10 characterized in that the at least one heating unit comprises at least one gas-gas-heat exchanger.

12. The device according to claim 10 characterized in that the heating element of the reactor is a gas burner or a fuel cell.

13. The device according to claim 10 characterized in that the at least two separation units for the separation of gases comprise:
a) at least one low temperature distillation unit and at least one olefin selective membrane, or
b) at least one hydrogen selective membrane and one olefin selective membrane.

14. Use of the catalyst of claim 1 for the preparation of olefins from C2-, C3- or C4-alkanes or a mixture thereof characterized in that the catalyst comprises:
a) at least one metal compound selected from a group consisting of metal carbide, -nitride, -silicide, -phosphide and -sulfide or mixtures thereof, wherein the metal is selected from a group consisting of molybdenum, tungsten, tantalum, vanadium, titanium, niobium, lanthanum and chromium, and
b) at least one non-Brønsted-acidic binder selected from a group consisting of AlPO₄, Bentonite, AlN and N₄Si₃.

15. The use according to claim 14 characterized in that the olefins are ethene and propene.
